SEQUENCE LISTING (59 sequences)

SEQ ID NO: 1 - protein, 15 aa, synthetic construct
  REGION 1..15: note = Synthetic
  SITE 1: note = misc_feature - Xaa can be any naturally occurring amino acid
  SITE 4: note = misc_feature - Xaa can be any naturally occurring amino acid
XWLXRALIKRIQAZI

SEQ ID NO: 2 - protein, 15 aa, synthetic construct
  REGION 1..15: note = Synthetic
  SITE 8: note = misc_feature - Xaa can be any naturally occurring amino acid
  SITE 14: note = misc_feature - Xaa can be any naturally occurring amino acid
RZLPQLVXRLVLRXS

SEQ ID NO: 3 - protein, 4 aa, synthetic construct
  REGION 1..4: note = Synthetic
PKGG

SEQ ID NO: 4 - protein, 4 aa, synthetic construct
  REGION 1..4: note = Synthetic
DATK

SEQ ID NO: 5 - protein, 7 aa, synthetic construct
  REGION 1..7: note = Synthetic
FPIPLPY

SEQ ID NO: 6 - protein, 78 aa, synthetic construct
  REGION 1..78: note = Synthetic
FPIPLPYCWLCRALIKRIQAMIPKGALAVAVAQVCRVVPLVAGGICQCLAERYSVILLDTLLGRMLPQLVCRLVLRCS

SEQ ID NO: 7 - protein, 41 aa, synthetic construct
  REGION 1..41: note = Synthetic
FPIPLPYCWLCRALIKRIQAMIPKGGRMLPQLVCRLVLRCS

SEQ ID NO: 8 - protein, 41 aa, synthetic construct
  REGION 1..41: note = Synthetic
FPIPLPYCWLCRALIKRIQAMIDATKRMLPQLVCRLVLRCS

SEQ ID NO: 9 - protein, 34 aa, synthetic construct
  REGION 1..34: note = Synthetic
CWLCRALIKRIQAMIPKGGRMLPQLVCRLVLRCS

SEQ ID NO: 10 - protein, 34 aa, synthetic construct
  REGION 1..34: note = Synthetic
CWLCRALIKRIQAMIDATKRMLPQLVCRLVLRCS

SEQ ID NO: 11 - protein, 15 aa, synthetic construct
  REGION 1..15: note = Synthetic
YWLYRALIKRIQALI

SEQ ID NO: 12 - protein, 15 aa, synthetic construct
  REGION 1..15: note = Synthetic
LWLYRALIKRIQALI

SEQ ID NO: 13 - protein, 15 aa, synthetic construct
  REGION 1..15: note = Synthetic
AWLYRALIKRIQALI

SEQ ID NO: 14 - protein, 15 aa, synthetic construct
  REGION 1..15: note = Synthetic
FWLYRALIKRIQALI

SEQ ID NO: 15 - protein, 15 aa, synthetic construct
  REGION 1..15: note = Synthetic
YWLFRALIKRIQALI

SEQ ID NO: 16 - protein, 15 aa, synthetic construct
  REGION 1..15: note = Synthetic
LWLFRALIKRIQALI

SEQ ID NO: 17 - protein, 15 aa, synthetic construct
  REGION 1..15: note = Synthetic
AWLFRALIKRIQALI

SEQ ID NO: 18 - protein, 15 aa, synthetic construct
  REGION 1..15: note = Synthetic
FWLFRALIKRIQALI

SEQ ID NO: 19 - protein, 15 aa, synthetic construct
  REGION 1..15: note = Synthetic
RLLPQLVYRLVLRYS

SEQ ID NO: 20 - protein, 15 aa, synthetic construct
  REGION 1..15: note = Synthetic
RLLPQLVYRLVLRLS

SEQ ID NO: 21 - protein, 15 aa, synthetic construct
  REGION 1..15: note = Synthetic
RLLPQLVYRLVLRAS

SEQ ID NO: 22 - protein, 15 aa, synthetic construct
  REGION 1..15: note = Synthetic
RLLPQLVYRLVLRFS

SEQ ID NO: 23 - protein, 15 aa, synthetic construct
  REGION 1..15: note = Synthetic
RLLPQLVFRLVLRYS

SEQ ID NO: 24 - protein, 15 aa, synthetic construct
  REGION 1..15: note = Synthetic
RLLPQLVFRLVLRLS

SEQ ID NO: 25 - protein, 15 aa, synthetic construct
  REGION 1..15: note = Synthetic
RLLPQLVFRLVLRAS

SEQ ID NO: 26 - protein, 15 aa, synthetic construct
  REGION 1..15: note = Synthetic
RLLPQLVFRLVLRFS

SEQ ID NO: 27 - protein, 41 aa, synthetic construct
  REGION 1..41: note = Synthetic
FPIPLPYYWLYRALIKRIQALIPKGGRLLPQLVYRLVLRYS

SEQ ID NO: 28 - protein, 41 aa, synthetic construct
  REGION 1..41: note = Synthetic
FPIPLPYLWLYRALIKRIQALIPKGGRLLPQLVYRLVLRLS

SEQ ID NO: 29 - protein, 41 aa, synthetic construct
  REGION 1..41: note = Synthetic
FPIPLPYAWLYRALIKRIQALIPKGGRLLPQLVYRLVLRAS

SEQ ID NO: 30 - protein, 41 aa, synthetic construct
  REGION 1..41: note = Synthetic
FPIPLPYFWLFRALIKRIQALIPKGGRLLPQLVFRLVLRFS

SEQ ID NO: 31 - protein, 41 aa, synthetic construct
  REGION 1..41: note = Synthetic
FPIPLPYLWLFRALIKRIQALIPKGGRLLPQLVFRLVLRLS

SEQ ID NO: 32 - protein, 41 aa, synthetic construct
  REGION 1..41: note = Synthetic
FPIPLPYAWLFRALIKRIQALIPKGGRLLPQLVFRLVLRAS

SEQ ID NO: 33 - protein, 41 aa, synthetic construct
  REGION 1..41: note = Synthetic
FPIPLPYYWLFRALIKRIQALIPKGGRLLPQLVFRLVLRYS

SEQ ID NO: 34 - protein, 41 aa, synthetic construct
  REGION 1..41: note = Synthetic
FPIPLPYFWLYRALIKRIQALIPKGGRLLPQLVYRLVLRFS

SEQ ID NO: 35 - protein, 41 aa, synthetic construct
  REGION 1..41: note = Synthetic
FPIPLPYYWLYRALIKRIQALIDATKRLLPQLVYRLVLRYS

SEQ ID NO: 36 - protein, 41 aa, synthetic construct
  REGION 1..41: note = Synthetic
FPIPLPYLWLYRALIKRIQALIDATKRLLPQLVYRLVLRLS

SEQ ID NO: 37 - protein, 41 aa, synthetic construct
  REGION 1..41: note = Synthetic
FPIPLPYAWLYRALIKRIQALIDATKRLLPQLVYRLVLRAS

SEQ ID NO: 38 - protein, 41 aa, synthetic construct
  REGION 1..41: note = Synthetic
FPIPLPYFWLFRALIKRIQALIDATKRLLPQLVFRLVLRFS

SEQ ID NO: 39 - protein, 41 aa, synthetic construct
  REGION 1..41: note = Synthetic
FPIPLPYLWLFRALIKRIQALIDATKRLLPQLVFRLVLRLS

SEQ ID NO: 40 - protein, 41 aa, synthetic construct
  REGION 1..41: note = Synthetic
FPIPLPYAWLFRALIKRIQALIDATKRLLPQLVFRLVLRAS

SEQ ID NO: 41 - protein, 41 aa, synthetic construct
  REGION 1..41: note = Synthetic
FPIPLPYYWLFRALIKRIQALIDATKRLLPQLVFRLVLRYS

SEQ ID NO: 42 - protein, 41 aa, synthetic construct
  REGION 1..41: note = Synthetic
FPIPLPYFWLYRALIKRIQALIDATKRLLPQLVYRLVLRFS

SEQ ID NO: 43 - protein, 34 aa, synthetic construct
  REGION 1..34: note = Synthetic
YWLYRALIKRIQALIPKGGRLLPQLVYRLVLRYS

SEQ ID NO: 44 - protein, 34 aa, synthetic construct
  REGION 1..34: note = Synthetic
LWLYRALIKRIQALIPKGGRLLPQLVYRLVLRLS

SEQ ID NO: 45 - protein, 34 aa, synthetic construct
  REGION 1..34: note = Synthetic
AWLYRALIKRIQALIPKGGRLLPQLVYRLVLRAS

SEQ ID NO: 46 - protein, 34 aa, synthetic construct
  REGION 1..34: note = Synthetic
FWLFRALIKRIQALIPKGGRLLPQLVFRLVLRFS

SEQ ID NO: 47 - protein, 34 aa, synthetic construct
  REGION 1..34: note = Synthetic
LWLFRALIKRIQALIPKGGRLLPQLVFRLVLRLS

SEQ ID NO: 48 - protein, 34 aa, synthetic construct
  REGION 1..34: note = Synthetic
AWLFRALIKRIQALIPKGGRLLPQLVFRLVLRAS

SEQ ID NO: 49 - protein, 34 aa, synthetic construct
  REGION 1..34: note = Synthetic
YWLFRALIKRIQALIPKGGRLLPQLVFRLVLRYS

SEQ ID NO: 50 - protein, 34 aa, synthetic construct
  REGION 1..34: note = Synthetic
FWLYRALIKRIQALIPKGGRLLPQLVYRLVLRFS

SEQ ID NO: 51 - protein, 34 aa, synthetic construct
  REGION 1..34: note = Synthetic
YWLYRALIKRIQALIDATKRLLPQLVYRLVLRYS

SEQ ID NO: 52 - protein, 34 aa, synthetic construct
  REGION 1..34: note = Synthetic
LWLYRALIKRIQALIDATKRLLPQLVYRLVLRLS

SEQ ID NO: 53 - protein, 34 aa, synthetic construct
  REGION 1..34: note = Synthetic
AWLYRALIKRIQALIDATKRLLPQLVYRLVLRAS

SEQ ID NO: 54 - protein, 34 aa, synthetic construct
  REGION 1..34: note = Synthetic
FWLFRALIKRIQALIDATKRLLPQLVFRLVLRFS

SEQ ID NO: 55 - protein, 34 aa, synthetic construct
  REGION 1..34: note = Synthetic
LWLFRALIKRIQALIDATKRLLPQLVFRLVLRLS

SEQ ID NO: 56 - protein, 34 aa, synthetic construct
  REGION 1..34: note = Synthetic
AWLFRALIKRIQALIDATKRLLPQLVFRLVLRAS

SEQ ID NO: 57 - protein, 34 aa, synthetic construct
  REGION 1..34: note = Synthetic
YWLFRALIKRIQALIDATKRLLPQLVFRLVLRYS

SEQ ID NO: 58 - protein, 34 aa, synthetic construct
  REGION 1..34: note = Synthetic
FWLYRALIKRIQALIDATKRLLPQLVYRLVLRFS

SEQ ID NO: 59 - protein, 41 aa, synthetic construct; identical to SEQ ID NO: 8
  REGION 1..41: note = Synthetic

(19) United States
(12) Patent Application Publication
(10) Pub. No.: US 2026/0345742 A1
(43) Pub. Date: Nov. 5, 2026

(54) SYNTHETIC SURFACTANTS FOR INHIBITING CORONAVIRUS INFECTION

(71) Applicant: Lundquist Institute for Biomedical Innovation at Harbor-UCLA Medical Center, Torrance, CA (US)

(72) Inventors: Frans J. WALTHER, Torrance, CA (US); Alan J. WARING, Torrance, CA (US); Larry M. GORDON, Torrance, CA (US)

(21) Appl. No.: 19/107,435

(22) PCT Filed: Aug. 28, 2023
(86) PCT No.: PCT/US2023/072984

§ 371 Date: Feb. 27, 2025

Related U.S. Application Data
(60) Provisional application No. 63/401,833, filed on Aug. 29, 2022.

(51) Int. Cl. A61K 38/17

(52) U.S. Cl. CPC A61K 38/395

(57) ABSTRACT

Compositions and methods are described that are useful for treating and preventing viral infections, such as those by SARS-CoV-2. It is discovered herein that synthetic surfactants such as SMB (SEQ ID NO:7) and B-YL (SEQ ID NO:27) can bind to SARS-CoV-2 Receptor Binding Domain (RBD) and the ACE2 receptor, thereby inhibiting the interaction between the virus and its target cells in the lung, minimizing the infectivity of the virus.

### CROSS REFERENCE TO RELATED APPLICATIONS

This application claims the benefit under 35 U.S.C. § 119(e) of the U.S. Provisional Application Ser. No. 63/401,833, filed Aug. 29, 2022, the content of which is hereby incorporated by reference in its entirety.

### REFERENCE TO AN ELECTRONIC SEQUENCE LISTING

The contents of the electronic sequence listing (339850.xml; Size: 76,700 bytes; and Date of Creation: Aug. 16, 2023) is herein incorporated by reference in its entirety.

### BACKGROUND

The COVID-19 pandemic has resulted in hundreds of millions of infected patients, with disease severity ranging from mild flu-like symptoms to severe acute respiratory distress syndrome (ARDS) and death. In humans, infection with the SARS-CoV-2 virus can lead to an acute viral infection with a mean incubation time of around 5 days. Clinical symptoms include fever, cough, fatigue, muscle pain, and dyspnea. A subsequent rapid worsening of respiratory problems may require invasive mechanical ventilation with oxygen supplementation and an administration of antivirals, monoclonal antibody therapy, and anti-coagulants.

In humans, membrane-bound protein angiotensin-converting Enzyme 2 is expressed in the epithelium of the lungs, small intestines, heart, liver, and kidneys. In the lungs, ACE2 is mainly present on the membranes of alveolar epithelial Type 2 cells. Binding of the receptor-binding domain (RBD) of the SARS-CoV-2 Spike 1 (S1) protein to ACE2 plays an essential role in viral entry and results in damage to the alveoli. Following infection, SARS-CoV-2 replicates in the cells of the respiratory and intestinal epithelium, leading to tissue damage and associated clinical symptoms.

Improved treatments of COVID-19 and its symptoms are needed.

### SUMMARY

It is discovered herein that synthetic surfactants containing the presently disclosed peptides, exemplified by SMB (SEQ ID NO:7) and B-YL (SEQ ID NO:27), can bind to SARS-CoV-2 Receptor Binding Domain (RBD) and the ACE2 receptor, thereby inhibiting the interaction between the virus and its target cells in the lung, minimizing the infectivity of the virus.

The present disclosure provides peptides that have anti-viral activity against SARS-CoV-2 in the lung, as well as other coronaviruses. Surfactants that contain such peptides, and related compositions, methods of preparing and using the compositions are also described. In one embodiment, the peptide includes an N-terminal helix, connected optionally through a turn, to a C-terminal helix of the α-helix of surfactant protein (SP)-B. The N-terminal or C-terminal helix can be modified, as compared to the natural SP-B peptide, with one or more substitutions of the cysteine and/or methionine residues or adding ion pair residue substitutions. In some embodiments, the turn is a natural or designer loop peptide sequence that facilitates formation of a helix-turn-helix structure. Example sequences are provided below.

A first aspect of the present invention relates to surface active peptides that bind with high affinity to the SARS-CoV-2 Receptor Binding Domain and the ACE2 receptor in a manner that block the virus from entering the host cell.

In one embodiment, the surfactant peptide comprises (i) a first fragment comprising the amino acid sequence of XWLXRALIKRIQAZI (SEQ ID NO: 1) or a first amino acid sequence having at least 90% sequence identity to SEQ ID NO: 1 and (ii) a second fragment comprising the amino acid sequence of RZLPQLVXRLVLRXS (SEQ ID NO: 2) or a second amino acid sequence having at least 90% sequence identity to SEQ ID NO: 2, wherein X and Z each can be any amino acid.

In some embodiments, X is any amino acid but at least one amino acid at the X positions is not cysteine. In some embodiments, Z is any amino acid but at least one amino acid at the Z positions is not methionine.

In some aspects, the peptide further comprises (iii) a turn between the first fragment and the second fragment. In some aspects, the turn comprises PKGG (SEQ ID NO:3). In some aspects, the turn can form a salt bridge between amino acids within the turn or between the turn and the first or second fragment. In some aspects, the turn comprises DATK (SEQ ID NO:4).

In some aspects, the first fragment is at the N-terminal end of the second fragment. In some aspects, the peptide further comprises an insertion sequence at the N-terminal end of the first fragment. In some aspects, the insertion sequence comprises FPIPLPY (SEQ ID NO:5).

In some aspects, the peptide is 100 amino acids in length or shorter. In some aspects, the peptide is 80 amino acids in length or shorter.

In some aspects, at least one amino acid at the X positions is not cysteine. In some aspects, each amino acid at the X positions is not cysteine. In some aspects, the amino acid at each X position is selected from the group consisting of Y, L, A, and F.

In some aspects, at least one amino acid at the Z positions is not methionine. In some aspects, each amino acid at the Z position is not methionine. In some aspects, the amino acid at each X position is leucine.

In some aspects, the first fragment comprises any amino acid sequence of SEQ ID NO: 11-18, an amino acid sequence having at least 90% sequence identity to any amino acid sequence of SEQ ID NO:11-18, or an amino acid sequence derived from any amino acid sequence of SEQ ID NO: 11-18 with one, two or three amino acid addition, deletion and/or substitution.

In some aspects, the second fragment comprises any amino acid sequence of SEQ ID NO: 19-26, an amino acid sequence having at least 90% sequence identity to any amino acid sequence of SEQ ID NO:19-26, or an amino acid sequence derived from any amino acid sequence of SEQ ID NO: 19-26 with one, two or three amino acid addition, deletion and/or substitution.

In some aspects, the peptide comprises any amino acid sequence of SEQ ID NO: 27-58, an amino acid sequence having at least 90% sequence identity to any amino acid sequence of SEQ ID NO:27-58, or an amino acid sequence derived from any amino acid sequence of SEQ ID NO: 27-58 with one, two or three amino acid addition, deletion and/or substitution.

Also provided, in one embodiment, is a composition comprising a peptide of the present disclosure and one or more phospholipid. In some aspects, the one or more phospholipid is selected from the group consisting of dipalmitoylphosphatidylcholine (DPPC), palmitoylolcoylphosphatidylcholine (POPC), phosphatidylglycerol (PG), palmitoyloleoylphosphatidylglycerol (POPG), cholesterol (Chol), 1,2-Dioleoyl-sn-glycero-3-phosphocholine (DOPC), 1-Palmitoyl-2-oleoyl-sn-glycero-3-phosphoethanolamine (POPE), 1-palmitoyl-2-oleoylsn-glycero phosphocholine (POPS), 1,2-Distearoyl-sn-glycero-3-phosphocholine (DSPC), 1,2-Dipalmitoyl-sn-glycero-3-phosphoethanolamine (DPPE), 1,2-dipalmitoyl-sn-glycero-3-phosphoglycerol (DPPG), a diether phosphonolipid analog of DPPC (DEPN-8), C16:0, C16:1 diether phosphonoglycerol (PG-1) and combinations thereof.

In some aspects, the one or more phospholipid comprises DPPC, POPC and POPG. In some aspects, the DPPC, POPC and POPG are at ratio of about (4-6):(2-4):(1-3).

Also provided, in one embodiment, is a method of inhibiting infection by a virus in a subject in need thereof, comprising administration to the patient a composition of the present disclosure. In some embodiments, the virus is a coronavirus. In some embodiments, the coronavirus is SARS-CoV-2, SARS-CoV, Middle East respiratory syndrome-related coronavirus (MERS-CoV). In some embodiments, the virus is an influenza virus. In some embodiments, the influenza virus is the H5N1 influenza A virus or the H7N9 influenza A virus.

### BRIEF DESCRIPTION OF THE DRAWINGS

FIG. 1. Molecular illustration of SARS-CoV-2 bound to synthetic surfactant B-YL peptide. B-YL backbone and aromatic side chains are highlighted in red. SARS-CoV-2 protein backbone is green and the RBD domain with interactive aromatic amino acid side chains is highlighted in blue. Tyr-8 of B-YL paired with Tyr-473 of the RBD, Tyr-11 of B-YL paired with Tyr-489 RBD, Tyr-34 of B-YL interacted with Tyr-495 and Phe-497 of the RBD, and Tyr-40 of B-YL interacted with Tyr-490 RBD.

FIG. 2. Molecular illustration of B-YL bound to the ACE2 lung cell receptor. The interactive B-YL backbone with aromatic and ionic residue side chains is shown in red. The ACE2 protein backbone is highlighted in green, while the interactive domain residucs with aromatic and ionic side chains is shown in blue. Ion pairs are shown between ACE2 Glu-30 and B-YL Arg-12, while hydrophobic ring interactions are labeled between ACE2 Tyr-83 and B-YL Phe-1.

FIG. 3. Surface plasmon resonance (SPR) sensorgrams of rbCovid-19-RBD protein construct and human ACE2 receptor binding to immobilized B-YL peptide. The B-YL peptide was biotinylated to facilitate attachment to the Biacore chip as described in Methodology. Solutions of 1 μM recombinant protein in HBS-EP buffer (10 mM Hepes, pH 7.4, 150 mM NaCl, 3 mM EDTA, 0.005% surfactant P20) were then flowed over the respective chip-linked peptides. The concentration dependent binding of the viral RBD construct (A) and the human ACE-2 receptor (B) to the B-YL lung surfactant peptide are shown with the SPR response indicated in relative response units (RU) on the graph's Y-axis.

FIG. 4. Surface plasmon resonance (SPR) sensor grams of SMB (upper curve) and B-YL (lower curve) peptide binding to the rhACE2 receptor protein. Solutions of 1 μM of recombinant protein in HBS-EP buffer were then flowed over the respective chip-linked peptides. The SPR responses are in relative response units (RU) on the Y axis.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a peptide” includes a plurality of peptides.

### 1. Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As used herein, the term “sequence identity” refers to a level of amino acid residue or nucleotide identity between two peptides or between two nucleic acid molecules. When a position in the compared sequence is occupied by the same base or amino acid, then the molecules are identical at that position. A peptide (or a polypeptide or peptide region) has a certain percentage (for example, at least about 60%, or at least about 65%, or at least about 70%, or at least about 75%, or at least about 80%, or at least about 83%, or at least about 85%, or at least about 90%, or at least about 95%, or at least about 98% or at least about 99%) of “sequence identity” to another sequence means that, when aligned, that percentage of bases (or amino acids) are the same in comparing the two sequences. It is noted that, for any sequence (“reference sequence”) disclosed in this application, sequences having at least about 60%, or at least about 65%, or at least about 70%, or at least about 75%, or at least about 80%, or at least about 83%, or at least about 85%, or at least about 90%, or at least about 95%, or at least about 98% or at least about 99% sequence identity to the reference sequence are also within the disclosure.

Likewise, the present disclosure also includes sequences that have one, two, three, four, or five substitution, deletion or addition of amino acid residues or nucleotides as compared to the reference sequences.

In any of the embodiments described herein, analogs of a peptide comprising any amino acid sequence described herein are also provided, which have at least about 80%, or at least about 83%, or at least about 85%, or at least about 90%, or at least about 95%, or at least about 98%, or at least about 99% sequence identity to any of reference amino acid sequences. In some embodiments, the analogs include one, two, three, four, or five substitution, deletion or addition of amino acid residues as compared to the reference sequences. In some embodiments, the substitution is a conservative substitution.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in Table A.

TABLE A

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me—Cys, Met, D-Met, Thr, |
| | D-Thr, L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me—Cys, Ile, D-Ile, Leu, |
| | D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

As used herein, the term “composition” refers to a preparation suitable for administration to an intended patient for therapeutic purposes that contains at least one pharmaceutically active ingredient, including any solid form thereof. The composition may include at least one pharmaceutically acceptable component to provide an improved formulation of the peptide, such as a suitable carrier. In certain embodiments, the composition is formulated as a film, gel, patch, or liquid solution.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the internal surface of the lung.

### 2. Surfactant Peptides for Treating or Preventing Viral Infections

COVID-19 pneumonia is initiated by the binding of the viral receptor-binding domain (RBD) of SARS-CoV-2 to the cellular receptor angiotensin-converting enzyme 2 (ACE2). Inflammation and tissue damage then lead to loss and dysfunction of surface activity that can be relieved by treatment with an exogenous lung surfactant.

The instant inventors studied the binding of two synthetic surfactant protein B (SP-B) peptide mimics, Super Mini-B (SMB, SEQ ID NO:7) and B-YL (SEQ ID NO:27), to a recombinant human ACE2 receptor protein construct using molecular docking and surface plasmon resonance (SPR) to evaluate their potential as antiviral drugs. The SPR measurements confirmed that both the SMB and B-YL peptides bind to the rhACE2 receptor with affinities like that of the viral RBD-ACE2 complex. These findings indicate that synthetic lung surfactant peptide mimics can act as competitive inhibitors of the binding of viral RBD to the ACE2 receptor.

Accordingly, one embodiment of the present disclosure provides a method for preventing, inhibiting or treating the infection of a virus in a subject in need thereof, or treating the infection or an associated symptom in the subject. In some embodiments, the method entails administering to the subject a surfactant peptide, such as any one of the SP-B peptide mimics as disclosed herein.

In one embodiment, the surfactant peptide includes an N-terminal helix, connected optionally through a turn, to a C-terminal helix of the alpha helix of surfactant protein (SP)-B. The N-terminal or C-terminal helix can be modified, as compared to the natural SP-B peptide, with one or more substitutions at the cysteine and/or methionine residues. In some embodiments, the turn is a natural or designer loop peptide sequence that facilitates formation of a helix-turn-helix structure.

The sequence of the alpha-helix of SP-B is provided in Table B (SEQ ID NO:6), where the N-terminal helix and the C-terminal helix are underlined. Two example peptides that include these helices are also listed in Table B, short-named “Mini-B or MB” (SEQ ID NO:9) and “Super Mini-B or SMB” (SEQ ID NO:7). In addition to the helices, Mini-B further includes a “PKGG” turn (SEQ ID NO:3). Super Mini-B then further includes the “insertion sequence” (SEQ ID NO: 5) from the natural SP-B peptide.

The Mini-B and Super Mini-B peptides can be modified by replacing the PKGG turn with another turn, such as DATK (SEQ ID NO: 4) which is discovered to be able to increase molecular stability and improve the ease of synthesis, folding and purification of the peptides. Example analogs in this respect include SMB-DATK (SEQ ID NO:8) and MB-DATK (SEQ ID NO: 10).

In some embodiments, any of these amino acid sequences can further be modified within either or both the helix regions. In one embodiment, at least one, two, three, or four, or all of the cysteines in the helix is substituted with another amino acid. In one embodiment, at least one cysteine in each helix is substituted wither another amino acid. In one embodiment, at least one of the helices has no cysteine residue. In one embodiment, the entire peptide includes no cysteine. In some embodiments, the substitution is with Y, L, A, or F.

Surprisingly, it is discovered that, even when the cysteines are substituted resulting in removal of the disulfide bonds, the peptide can still form a desired helix-turn-helix structure and is more stable and effective. In some examples, when the cysteines are substituted with one or more tyrosine residues, the hydrophobic core formed by the tyrosine residues can further help stabilize the peptide.

In one embodiment, at least one of the methionine residues is substituted with another amino acid. In one embodiment, both of the methionine residues are substituted. In some embodiments, the substitution is with leucine. Also surprisingly, such a substitution does not change the structure of the peptide but rather makes it more stable and easier to fold and manufacture. Further, the removal of methionine renders the peptide resisting oxidative stress.

In one embodiment, the surfactant peptide includes (i) a first fragment comprising the amino acid sequence of XWLXRALIKRIQAZI (SEQ ID NO: 1) or a first amino acid sequence having at least 90% (or at least 80%, 85% or 95%) sequence identity to SEQ ID NO: 1 and (ii) a second fragment comprising the amino acid sequence of RZLPQLVXRLVLRXS (SEQ ID NO: 2) or a second amino acid sequence having at least 90% (or at least 80%, 85% or 95%) sequence identity to SEQ ID NO: 2, wherein X is any amino acid and Z is any amino acid.

In some embodiments, X is any amino acid but at least one amino acid at the X positions is not cysteine. In some embodiments, Z is any amino acid but at least one amino acid at the Z positions is not methionine. Non-limiting examples of SEQ ID NO:1 include SEQ ID NO:11-18. Non-limiting examples of SEQ ID NO:2 include SEQ ID NO:19-26.

In some embodiments, the peptide further includes a turn between the first fragment and the second fragment. A “turn” as used herein, refers to a relatively short (e.g., less than 50 amino acids in length) amino acid fragment that forms a secondary structure in a polypeptide chain where the polypeptide chain reverses its overall direction. Examples of turns include, without limitation, α-turns, β-turns, γ-turns, δ-turns, π-turns, loops, multiple turns and hairpins. The turn is typically from one amino acid to about 50 amino acids (or to about 45, 40, 35, 30, 25, 20, 15, 10, 9, 8, 7, 6 or 5 amino acids) in length. In some embodiment, the turn does not include cysteine. In some embodiments, the turn does not include methionine.

In some embodiments, the turn includes an amino acid that forms a salt bridge with either of the helices. In some embodiments, the turn includes amino acids to form a salt bridge within.

Non-limiting examples of turns include PKGG (SEQ ID NO:3), DATK (SEQ ID NO:4) and amino acids 23-63 of SEQ ID NO:6 or a portion or combination of portions thereof.

It is contemplated that the helices can be orientated either way. In one embodiment, SEQ ID NO: 1 (or the first fragment) can be at the N-terminal direction of SEQ ID NO:2 (or the second fragment). In one embodiment, SEQ ID NO:1 (or the first fragment) can be at the C-terminal direction of SEQ ID NO:2 (or the second fragment).

In some embodiments, the surfactant peptide further includes an insertion sequence at the N-terminal end of the peptide. In some embodiments, the peptide further includes an insertion sequence at the N-terminal direction of the first fragment or the N-terminal direction of the second fragment. The insertion sequence, in some embodiments, includes at least one proline. In another embodiment, the insertion sequence includes at least a leucine or isoleucine. A non-limiting example of the insertion sequence is FPIPLPY (SEQ ID NO:5).

The total length of the surfactant peptide varies from 20 amino acids to about 100 amino acids. In one embodiment, the peptide is not longer than about 100, or 90, 80, 70, 60 or 50 amino acids long.

Non-limiting examples of the surfactant peptide include SEQ ID NO: 7-10 and 59 or an amino acid sequence having at least 90% (or at least 80%, 85% or 95%) sequence identity to any amino acid sequence of SEQ ID NO: 7-10 and 59, or an amino acid sequence derived from any amino acid sequence of SEQ ID NO: 7-10 and 59 with one, two or three amino acid addition, deletion and/or substitution.

Non-limiting examples of the surfactant peptide include SEQ ID NO: 27-58 or an amino acid sequence having at least 90% (or at least 80%, 85% or 95%) sequence identity to any amino acid sequence of SEQ ID NO:27-58, or an amino acid sequence derived from any amino acid sequence of SEQ ID NO: 27-58 with one, two or three amino acid addition, deletion and/or substitution.

This surfactant peptides are shown to have dual clinical roles in alleviating surfactant insufficiency and inhibition of binding of the SARS-CoV-2 Receptor Binding Domain to the ACE2 receptor on the host cells.

Table B below lists the amino acid sequences, SEQ ID NOs and, in some cases, short names for various peptides disclosed in the present application.

TABLE B
Peptide Sequences and Names

SEQ ID NO: 1 XWLXRALIKRIQAZI
SEQ ID NO: 2 RZLPQLVXRLVLRXS
SEQ ID NO: 3 PKGG
SEQ ID NO: 4 DATK
SEQ ID NO: 5 FPIPLPY
SEQ ID NO: 11 YWLYRALIKRIQALI
SEQ ID NO: 12 LWLYRALIKRIQALI
SEQ ID NO: 13 AWLYRALIKRIQALI
SEQ ID NO: 14 FWLYRALIKRIQALI
SEQ ID NO: 15 YWLFRALIKRIQALI
SEQ ID NO: 16 LWLFRALIKRIQALI
SEQ ID NO: 17 AWLFRALIKRIQALI
SEQ ID NO: 18 FWLFRALIKRIQALI
SEQ ID NO: 19 RLLPQLVYRLVLRYS
SEQ ID NO: 20 RLLPQLVYRLVLRLS
SEQ ID NO: 21 RLLPQLVYRLVLRAS
SEQ ID NO: 22 RLLPQLVYRLVLRES
SEQ ID NO: 23 RLLPQLVERLVLRYS
SEQ ID NO: 24 RLLPQLVFRLVLRLS
SEQ ID NO: 25 RLLPQLVFRLVLRAS
SEQ ID NO: 26 RLLPQLVFRLVLRES
Alpha-helix of SP-B:
FPIPLPYCWLCRALIKRIQAMIPKGALAVAVAQVCRVVPLVAGGICQCLAERYSVILLDTLLG
RMLPQLVCRLVLRCS
(SEQ ID NO: 6)
Super Mini-B: FPIPLPYCWLCRALIKRIQAMIPKGGRMLPQLVCRLVLRCS (SEQ ID NO: 7)
B-YL: FPIPLPYYWLYRALIKRIQALIPKGGRLLPQLVYRLVLRYS (SEQ ID NO: 27)
B-LYL: FPIPLPYLWLYRALIKRIQALIPKGGRLLPQLVYRLVLRLS (SEQ ID NO: 28)
B-AYL: FPIPLPYAWLYRALIKRIQALIPKGGRLLPQLVYRLVLRAS (SEQ ID NO: 29)
B-FFL: FPIPLPYFWLFRALIKRIQALIPKGGRLLPQLVFRLVLRES SEQ ID NO: 30)
B-LFL: FPIPLPYLWLFRALIKRIQALIPKGGRLLPQLVERLVLRLS (SEQ ID NO: 31)
B-AFL: FPIPLPYAWLFRALIKRIQALIPKGGRLLPQLVFRLVLRAS (SEQ ID NO: 32)
B-YFL: FPIPLPYYWLFRALIKRIQALIPKGGRLLPOLVERLVLRYS (SEQ ID NO: 33)
B-FYL: FPIPLPYFWLYRALIKRIQALIPKGGRLLPQLVYRLVLRFS (SEQ ID NO: 34)
SMB-DATK: FPIPLPYCWLCRALIKRIQAMIDATKRMLPQLVCRLVLRCS (SEQ ID NO: 8)
B-DATK-YL: FPIPLPYYWLYRALIKRIQALIDATKRLLPQLVYRLVLRYS (SEQ ID NO: 35)
B-DATK-LYL: FPIPLPYLWLYRALIKRIQALIDATKRLLPQLVYRLVLRLS (SEQ ID NO: 36)
B-DATK-AYL: FPIPLPYAWLYRALIKRIQALIDATKRLLPQLVYRLVLRAS (SEQ ID NO: 37)
B-DATK-FFL: FPIPLPYFWLFRALIKRIQALIDATKRLLPQLVERLVLRES (SEQ ID NO: 38)
B-DATK-LFL: FPIPLPYLWLFRALIKRIQALIDATKRLLPQLVFRLVLRLS (SEQ ID NO: 39)
B-DATK-AFL: FPIPLPYAWLFRALIKRIQALIDATKRLLPQLVFRLVLRAS (SEQ ID NO: 40)
B-DATK-YFL: FPIPLPYYWLFRALIKRIQALIDATKRLLPOLVERLVLRYS (SEQ ID NO: 41)
B-DATK-FYL: FPIPLPYFWLYRALIKRIQALIDATKRLLPQLVYRLVLRES (SEQ ID NO: 42)
Mini-B: CWLCRALIKRIQAMIPKGGRMLPQLVCRLVLRCS (SEQ ID NO: 9)
MB-YL: YWLYRALIKRIQALIPKGGRLLPQLVYRLVLRYS (SEQ ID NO: 43)
MB-LYL: LWLYRALIKRIQALIPKGGRLLPQLVYRLVLRLS (SEQ ID NO: 44)
MB-AYL: AWLYRALIKRIQALIPKGGRLLPQLVYRLVLRAS (SEQ ID NO: 45)
MB-FFL: FWLFRALIKRIQALIPKGGRLLPQLVFRLVLRES (SEQ ID NO: 46)
MB-LFL: LWLFRALIKRIQALIPKGGRLLPQLVERLVLRLS (SEQ ID NO: 47)
MB-AFL: AWLFRALIKRIQALIPKGGRLLPQLVERLVLRAS (SEQ ID NO: 48)
MB-YFL: YWLFRALIKRIQALIPKGGRLLPQLVFRLVLRYS (SEQ ID NO: 49)
MB-FYL: FWLYRALIKRIQALIPKGGRLLPQLVYRLVLRES (SEQ ID NO: 50)
MB-DATK: CWLCRALIKRIQAMIDATKRMLPQLVCRLVLRCS (SEQ ID NO: 10)
MB-DATK-YL: YWLYRALIKRIQALIDATKRLLPQLVYRLVLRYS (SEQ ID NO: 51)
MB-DATK-LYL: LWLYRALIKRIQALIDATKRLLPQLVYRLVLRLS (SEQ ID NO: 52)
MB-DATK-AYL: AWLYRALIKRIQALIDATKRLLPQLVYRLVLRAS (SEQ ID NO: 53)
MB-DATK-FFL: FWLFRALIKRIQALIDATKRLLPQLVFRLVLRES (SEQ ID NO: 54)
MB-DATK-LFL: LWLFRALIKRIQALIDATKRLLPQLVERLVLRLS (SEQ ID NO: 55)
MB-DATK-AFL: AWLFRALIKRIQALIDATKRLLPQLVFRLVLRAS (SEQ ID NO: 56)
MB-DATK-YFL: YWLFRALIKRIQALIDATKRLLPQLVFRLVLRYS (SEQ ID NO: 57)
MB-DATK-FYL: FWLYRALIKRIQALIDATKRLLPQLVYRLVLRES (SEQ ID NO: 58)
S-MM DATK: FPIPLPYCWLCRALIKRIQAMIDATKRMLPQLVCRLVLRCS (SEQ ID NO: 59)

### 3. Synthesis of Surfactant Peptides

The peptides described herein can be ordered from a commercial source or partially or fully synthesized using methods well known in the art (e.g., chemical and/or biotechnological methods). In certain embodiments, the peptides are synthesized according to solid phase peptide synthesis protocols that are well known in the art. In another embodiment, the peptide is synthesized on a solid support according to the well-known Fmoc protocol, cleaved from the support with trifluoroacetic acid and purified by chromatography according to methods known to persons skilled in the art. In other embodiments, the peptide is synthesized utilizing the methods of biotechnology that are well known to persons skilled in the art. In one embodiment, a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptidc), the plasmid is transfected into a host organism for expression, and the peptide is then isolated from the host organism or the growth medium, e.g., by affinity purification. Recombinant DNA technology methods are described in Sambrook et al., “Molecular Cloning: A Laboratory Manual”, 3rd Edition, Cold Spring Harbor Laboratory Press, (2001), incorporated herein by reference, and are well-known to the skilled biochemist.

The peptides can be also prepared by using recombinant expression systems. Generally, this involves inserting the nucleic acid molecule into an expression system to which the molecule is heterologous (i.e., not normally present). One or more desired nucleic acid molecules encoding a peptide of the disclosure may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The nucleic acid molecules can be derived from the known SP-B nucleotides. In certain embodiments, it may be desirable to prepare codon-enhanced nucleic acids that will favor expression of the desired peptide in the transgenic expression system of choice.

The preparation of the nucleic acid constructs can be carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. Other vectors are also suitable.

Once a suitable expression vector is selected, the desired nucleic acid sequences are cloned into the vector using standard cloning procedures in the art. The vector is then introduced to a suitable host.

Purified peptides may be obtained by several methods. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

Alternatively, if the peptide of interest is not secreted, it can be isolated from the recombinant cells using standard isolation and purification schemes. This includes disrupting the cells (e.g., by sonication, freezing, French press, etc.) and then recovering the peptide from the cellular debris. Purification can be achieved using the centrifugation, precipitation, and purification procedures described above.

Whether the peptide of interest is secreted or not, it may also contain a purification tag (such as poly-histidine, a glutathione-5-transferase, or maltose-binding protein (MBP−)), which assists in the purification but can later be removed, i.e., cleaved from the peptide following recovery. Protease-specific cleavage sites can be introduced between the purification tag and the desired peptide. The desired peptide product can be purified further to remove the cleaved purification tags.

### 4. Surfactant Compositions and Formulations

Surfactants and compositions that include any one or more of the peptides as disclosed herein are also provided. In one embodiment, the composition includes any one or more of the peptides and one or more phospholipid.

There are an abundance of kinds of phospholipids suitable for use in surfactants. Non-limiting examples include dipalmitoylphosphatidylcholine (DPPC), palmitoyloleoylphosphatidylcholine (POPC), phosphatidylglycerol (PG), palmitoyloleoylphosphatidylglycerol (POPG), cholesterol (Chol), glycerophospholipids such as 1,2-Dioleoyl-sn-glycero-3-phosphocholine (DOPC), 1-Palmitoyl-2-oleoyl-sn-glycero-3-phosphocthanolaminc (POPE), 1-palmitoyl-2-olcoylsn-glyccro phosphocholine (POPS), 1,2-Distearoyl-sn-glycero-3-phosphocholine (DSPC), 1,2-Dipalmitoyl-sn-glycero-3-phosphoethanolamine (DPPE), 1,2-dipalmitoyl-sn-glycero-3-phosphoglycerol (DPPG) and diether phosphonolipid analogs of DPPC and phosphatidylglycerol (e.g., DEPN-8 and PG-1).

The phospholipids can be mixed at suitable ratios, in some embodiments. For instance, DPPC:POPC:POPG can be used a ratio of about 5:3:2, DPPC:POPG at a ratio of about 7:3, DEPN-8:PG-1 at about 9:1 or 8:2. In a particular example, the phospholipids include DPPC, POPC and POPG. In one aspect, the DPPC, POPC and POPG are at ratio of about (4-6):(2-4):(1-3).

In various embodiments described herein, the peptides described herein can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced. Non-conservative substitutions may too be possible, provided that they do not substantially affect the binding activity of the peptide (i.e., collagen binding affinity).

The surfactant compositions can further include any one or more of a non-phospho surfactant. As used herein, the term “non-phospho surfactant” refers to surface active compounds that do not possess a phospho group (e.g., phosphate, phosphonate, etc.). Exemplary non-phospho surfactants include, without limitation, a free fatty acid, hexadecanol, or cholesterol.

Preferred free fatty acids include saturated and monounsaturated C₁₀ to C₂₄ hydrocarbons, more preferably C₁₂-C₂₀ hydrocarbons, most preferably C₁₄-C₁₈ hydrocarbons. Of these, saturated hydrocarbons are preferred.

The peptides or compositions of the present disclosure can be used for delivering pharmaceutical agents to a subject in need thereof. In one embodiment, the composition (or formulation) includes a peptide or composition of the earlier disclosure and a therapeutic agent. The therapeutic agent can be any agent that is shown, tested, or proposed to have therapeutic effects.

### Treatment Methods and Doses

Also provided, in one embodiment, is a method of preventing, inhibiting or treating infection by a virus in a subject in need thereof, or treating an associated symptom. In one embodiment, the method entails administration to the patient a composition of the present disclosure. In some embodiments, the virus is a coronavirus. In some embodiments, the coronavirus is SARS-CoV-2, SARS-CoV, Middle East respiratory syndrome-related coronavirus (MERS-CoV). In some embodiments, the virus is an influenza virus. In some embodiments, the influenza virus is the H5N1 influenza A virus or the H7N9 influenza A virus.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Peptides may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, the peptides disclosed herein may be used in combination with one or more additional therapeutic agent that are being used and/or developed to treat viral infections, such as nirmatrelvir/ritonavir (marketed as Paxlovid) for treating COVID-19. In some embodiments, the one or more additional therapeutic agent may be remdesivir.

In some embodiments, the one or more additional therapeutic agent may be favipiravir, fingolimod, and/or methylprednisolonc. In some embodiments, the one or more additional therapeutic agent may be bevacizumab. In some embodiments, the one or more additional therapeutic agent may be chloroquine phosphate, chloroquine, or hydroxychloroquine sulfate.

Surfactant peptides provided herein are usually administered in the form of pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

Aerosol delivery of the compositions can be accomplished with a vibrating mesh nebulizer. However, both liquid and dry-powder nebulizers can also be used. The type of nebulizer can vary and the drug combination can be nebulized as a wet or dry preparation.

Nebulizers and pressurized metered-dose inhalers can be used to deliver aerosolized drugs. Nebulizers transform liquid formulations and suspensions into medical aerosol and are understood to include jet nebulizers, ultrasonic nebulizers and mesh nebulizers. Mesh nebulizers uslower-frequency waves and eliminate the heating issues that can denature proteins during aerosolization.

Mesh nebulizers typically force liquid medications through multiple apertures in a mesh or aperture plate to generate aerosol. Mesh nebulizers are advantageous because they provide consistent aerosol generation and a predominately fine-particle fraction reaching into the peripheral lung, low residual volume, and the ability to nebulize in low drug volumes. The size of the pore, the aerosol chamber, and the reservoir, as well as the output rate of mesh nebulizers, can be adjusted for different drugs in order to optimize aerosol drug delivery to patients.

Mesh nebulizers can be classified as active mesh nebulizers and passive mesh nebulizers. Active mesh nebulizers use a piezo element that contracts and expands on application of an electric current and vibrates a precisely drilled mesh in contact with the medication to generate aerosol. One suitable mesh nebulizer for the compositions described herein is the Aeroneb (Acrogen, Galway, Ireland) nebulizers which can be used for both spontaneously breathing and ventilator-dependent patients.

The aerosolized composition can be administered using either a mouthpiece or a face mask, with a face mask being suitable for infants, small children, and patients with cognitive problems. The aerosolized composition can have a median mass aerodynamic diameter (MMAD) of 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. The MMAD may also be within a range between any two of the foregoing values.

The specific dose level of a peptide of the present application for any particular subject will depend upon a variety of factors including the activity of the specific peptide employed, the age, body weight, general health, sex, diet, time of administration, route of administration, and rate of excretion, drug combination and the severity of the particular disease in the subject undergoing therapy. For example, a dosage may be expressed as a number of milligrams of a peptide described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The peptides of the present application or the compositions thereof may be administered once, twice, three, or four times daily, using any suitable mode described above. Also, administration or treatment with the peptides may be continued for a number of days; for example, commonly treatment would continue for at least 7 days, 14 days, or 28 days, for one cycle of treatment.

### EXPERIMENTAL EXAMPLES

### Example 1. B-YL Binding to RBD and ACE2

B-YL-Surfactant is a synthetic lung surfactant that has been developed to treat mammalian lungs that either lack native lung surfactant, such as in premature infants with respiratory distress syndrome (RDS), or have less efficacy because of damaged endogenous lung surfactant, such as acute respiratory distress syndrome (ARDS). This surfactant formulation is composed of a single peptide, B-YL (SEQ ID NO:27), and emulates the lipid transport function of native Surfactant Protein B (SP-B) that facilitates rapid transport of lipids to and from the monolayer of phospholipids critical in maintaining lung functionality. The peptide-lipid formulation also contains several synthetic lipids that represent major phospholipid molecular species found in native lung surfactant, thereby optimizing emulation of native lung surfactant for replacement therapy. Some of the important advantages of this synthetic lung surfactant preparation are that it can be produced in large amounts very economically, has a very good shelf-life compared to preparations derived from animal sources, and can be administered to the lungs in various ways. These treatment approaches include an aqueous dispersion of the peptide-lipid material, as an aerosol spray, or as a dry powder.

In addition to the above advantages of synthetic surfactant formulations that use a synthetic SP-B peptide mimic in formulations with surfactant lipids targeted for replacement therapy, the B-YL family of peptides also has potential as antiviral therapy. Molecular simulations of B-YL docked to the Receptor Binding Domain (RBD) of the SARS-CoV-2 spike protein and the target host ACE2 receptor that is responsible for the entry of the virus into the host cells in the lung, indicate that B-YL peptide binds with high affinity to these target viral-receptor proteins. These findings were validated with in vitro experimental binding studies by surface plasmon resonance. These studies suggest that surfactant formulations containing B-YL peptide can act as a blocking inhibitor preventing viral entry in vivo in the lung.

In-Silico Molecular Modeling of B-YL Residue Specific Interactions with SARS-CoV-2 Spike Protein Receptor Binding Domain and the Interaction of B-YL with the ACE2 Receptor Domain

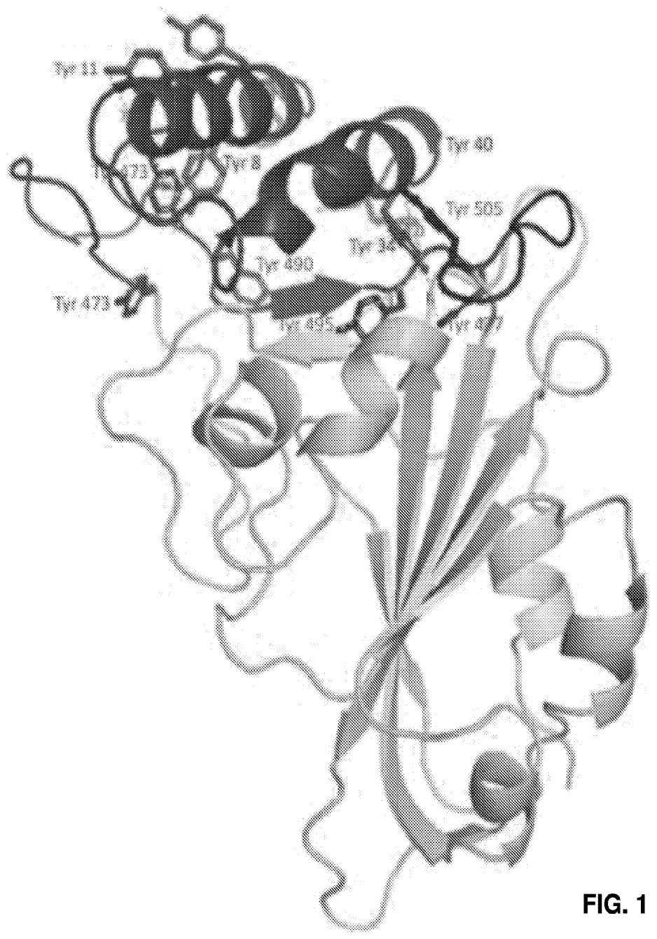

We tested whether B-YL could bind the RBD viral domain with high affinity. We used a miniprotein—spike protein structure from the Protein Data Bank and mutated the miniprotein structure to that of the B-YL surfactant peptide with PyMOL (Schrodinger LLC version 2.2.3). This preliminary docked B-YL-SARS-CoV-2 spike protein was then refined by the on-line docking program RosettaDock. The best fit protein complex for B-YL-SARS-CoV-2 spike protein of best fit ten RosettaDock ensembles was then subjected to molecular dynamics refinement using the Gromacs force field (version 2020) with the Charmm 36 parameter set in a simulated aqueous environment at 37 °C for 100 nano seconds. The final conformation is shown in FIG. 1.

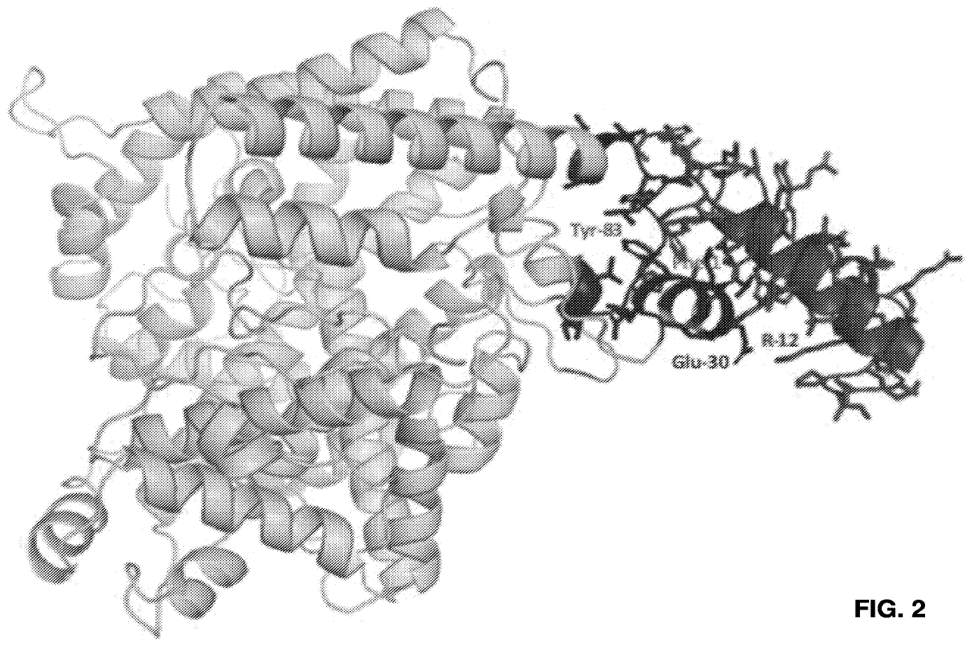

Visual inspection of the ensemble indicates that the B-YL surfactant peptide interacts with the RBD of the SARS-CoV-2 spike protein in the same helical orientation as the miniprotein and has multiple tyrosine interactions between the aromatic side chains of the bond peptide and SARS-CoV-2 protein. Similar docking-molecular dynamics simulations were carried out with B-YL and the human receptor protein ACE2 molecular coordinates (PDB accession code: 6M0J) that is the target receptor for mediation of the Covid-19 viral entry into lung alveolar cells. The final conformation for the B-YL-ACE2 complex after 100 nano-seconds is illustrated in FIG. 2. This simulation not only shows that there are aromatic side chain interactions between ACE2 and B-YL proteins, there is an additional strong ionic pair interaction between the anionic glutamic acid residue side chain and a cationic arginine residue side chain of the B-YL surfactant peptide suggesting a very stable binding of the lung receptor and the synthetic B-YL surfactant peptide.

Experimental Validation of in-Silico Predicted Binding of B-YL Lung Surfactant Peptide to SARS-CoV-2 RBD Domain

One of the best in vitro determinations of the binding of proteins and peptides to one another is by surface plasmon resonance (SPR). This technique allows the direct measurement of the binding of a peptide or protein by attaching one protein to a sensor chip surface and flowing a solution containing a second protein over the surface. The binding affinities derived from such measurements are very similar to those observed in vivo. The resulting sensorgram shows the time-course of association of the mobile phase protein with that of the sensor chip attached protein and directly measures the off and on rates of protein-protein interactions that can then be used to accurately determine protein binding affinities.

The binding affinity of the B-YL lung surfactant peptide to the coronavirus recombinant spike protein construct of SARS-CoV-2 (viral spike protein amino acid sequence) that included the Receptor Binding Domain (RBD) was measured with SPR spectroscopy using a Biacore T200 system (GE Healthcare Bio-Sciences Corp, Piscataway, NJ 08855). Because of limited aqueous solution solubility, the B-YL peptide was biotinylated to increase the peptide's specific interaction with series S Senor Chip SA (GE, BR100530). Binding measurements were then made by flowing a running buffer solution of the test in HBS-EP buffer (10 mM Hepes, pH 7.4, 150 mM NaCl, 3 mM EDTA, 0.005% surfactant P20) over the chip-associated peptide at a flow rate of 30 μl/min to determine the binding affinity at 37 °C.

B-YL peptide binding to the recombinant SARS-CoV-2 RBD domain dissolved in a series of seven concentrations (1,000, 500, 250, 125, 62.5, 31.25 and 0 nM) in the analyte and was determined from the sensorgrams, in which arbitrary response units (RUS) are recorded as a function of time. The binding associated with control medium containing no peptide was subtracted from final affinity traces. The mean “on” and “off” rate constants (kon and koff) and the dissociation equilibrium constant (KD=koff/kon) were calculated using Biacore Insight Evaluation Software based on curve fitting measurements.

Kinetic analysis of these traces to determine relative binding (affinity) of the B-YL peptide to the SARS-CoV-2 construct are summarized in Table 1. The SARS-CoV-2 RBD construct had a high affinity for the B-YL peptide having a high RU and low dissociation constant (KD) indicating a high affinity for the viral protein. These preliminary experimental binding results validate the interaction between B-YL and SARS-CoV-2 RBD domain as predicted from the in-silico modeling of these two protein constructs.

TABLE 1
Summary of the Affinity assay between
SARS-CoV-2 and biotinylated B-YL

| | Chip- | | | |
|---|---|---|---|---|
| Aqueous | associated | kₒₙ | k_{off} | KD |
| protein | peptide | (1/Ms) | (1/s) | (M) |
| SARS-CoV-2-RBD | B-YL | 8.9 × 10² | 5.08 × 10⁻² | 5.69 × 10⁻⁵ |

In Table 1, association and dissociation kinetic rate constants (kon, koff) and equilibrium dissociation constants (KD) were calculated from surface plasmon resonance (SPR) kinetic measurements for aqueous SARS-CoV-2 RBD expressed protein flowing past B-YL surfactant peptide on the Biacore chip. The recombinant SARS-CoV-2 RBD protein was dissolved in running buffer and was flowed past the sensor chip with a BiocoreT200 system (Methods). Kinetic rate constants and equilibrium dissociation constants were determined from curve fitting analysis of SPR traccs.

### Example 2. Surface Plasmon Resonance (SPR) Measurements of the Binding of B-YL Surfactant Peptide to Lung Receptor ACE-2 and Covid-19 RBC Domains

The binding affinities of the B-YL lung surfactant peptide to the Angiotensin-Converting Enzyme 2 (ACE-2) receptor recombinant construct (human amino acid sequence) and the coronavirus recombinant spike protein construct of Covid-19 (viral spike protein amino acid sequence) that included the Receptor Binding Domain (RBD) were measured with surface plasmon resonance (SPR) spectroscopy using a Biacore T200 system (GE Healthcare Bio-Sciences Corp, Piscataway, NJ 08855). Because of limited aqueous solution solubility, the B-YL peptide was biotinylated so to increase the peptides specific interaction with series S Senor Chip SA (GE, BR100530). Binding measurements were then made by flowing a running buffer solution of the test in HBS-EP buffer (i.e., 10 mM Hepes, pH 7.4, 150 mM NaCl, 3 mM EDTA, 0.005% surfactant P20) over the chip-associated peptide at a flow rate of 30 μl/min to determine the binding affinity at 37 °C. B-YL peptide binding to the recombinant ACE-2 receptor or the recombinant Covid-19 RBD domain dissolved in a series of seven concentrations (1000, 500, 250, 125, 62.5, and 31.25 nM) in the analyte and was determined from the sensorgrams, in which the arbitrary response units (RU) are recorded as a function of time. The binding associated with control medium containing no peptide was subtracted from final affinity traces. The mean “on” and “off” rate constants (kₒₙ and k_{off}) and the dissociation equilibrium constant (K_{D}=k_{off}/kₒₙ) was calculated using Biacore Insight Evaluation Software based on curve fitting measurements.

To better characterize the potential degree and type of interaction of lung ACE-2 and known viral spike protein construct with the lung surfactant peptide B-YL, we used surface plasmon resonance (SPR) to test the binding of the peptides to the lung surfactant peptide construct. The SPR binding study was accomplished by associating the ligand (B-YL peptide) immobilized on the SPR sensor surface, and the solute containing the ACE-2 or the Covid-19 protein construct was then flowed past the sensor linked molecule. The binding of the solute to the sensor surface ligand results in an evanescent sensor response, is measured in Response Units (RU) that are proportional to the bound mass. This technique has been shown to be very useful in the study of ligand binding in protein-protein interactions (Patching SG, BBA 2014; 1839:43-55).

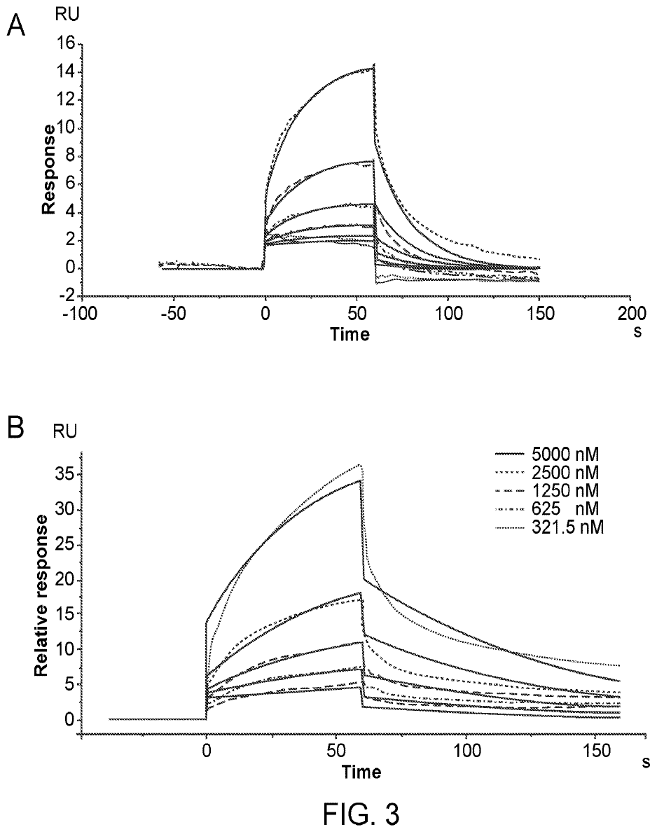

Representative sensorgrams for the interaction of the lung surfactant B-YL peptide tested in the present study with the ACE-2 and Covid-19 constructs are shown FIG. 3. Kinetic analysis of these traces to determine relative binding (affinities) of the peptides to the loop construct are summarized in Table 2. The ACE-2 construct had the highest binding affinity for the B-YL peptide having lowest dissociation constant (K_{D}). The viral RBD had a lower affinity with corresponding higher K_{D} values.

TABLE 2
Comparison of In Silico Binding free energy (ΔG) and
Equilibrium Binding Constants (K_{D}) with experimentally
determined binding data using Surface Plasmon Resonance.

| | | |
|---|---|---|
| Protein-Protein Interaction | ΔG (kcal mol⁻¹) | K_{D} (M) |

Binding Free Energies and Affinities of the binding of ACE2 -
Covid RBD from experimental SPR measurements compared
with that using structure-based prediction methodology

| | | |
|---|---|---|
| ACE2 - RBD (PDB: 6m0j.pdb) | −11.90 | 1.90 × 10⁻⁰⁹ |
| ACE2 - RBD (R&D Sys SPR Data)* | −11.82 | 7.04 × 10⁻⁰⁹ |

Binding Free Energies and Affinities Predicted from
Docking of Molecular Complexes (HADDOCK)

| | | |
|---|---|---|
| ACE2 - B-YL | −11.10 | 5.20 × 10⁻⁰⁹ |
| RBD(wt)- B-YL | −11.00 | 8.40 × 10⁻⁰⁹ |

Binding Free Energies and Affinities Predicted from Molecular
Simulations of Complexes to test ensemble stability

| | | |
|---|---|---|
| ACE2 (PDB 6M0J) - RBD(wt) | −5.00 | 2.30 × 10⁻⁰⁴ |
| ACE2- BYL | −3.10 | 5.60 × 10⁻⁰³ |
| RBD(wt) - B-YL | −4.50 | 5.40 × 10⁻⁰⁴ |

Experimentally Determined Binding Free Energies and
Affinities from SPR experimental measurements

| | | |
|---|---|---|
| ACE2 - B-YL- expt.data - SPR | −5.79 | 4.37 × 10⁻⁰⁶ |
| RBD(wt)- B-YL- expt.data - SPR | −5.80 | 5.70 × 10⁻⁰⁵ |

*RBD(wt)-ACE-2 SPR binding data determined by R&D Systems and listed in their spec sheet for the interaction of RBD(wt) construct with ACE-2 construct

TABLE 3
Surface Plasmon Resonance metrics derived from the time
course of binding of the ACE2 and RBD to the B-YL peptide

| Aqueous | Chip-linked | kₒₙ | k_{off} | K_{D} |
|---|---|---|---|---|
| peptide | peptide | (1/Ms) | (1/s) | (M) |
| ACE2 | B-YL | 2.98 × 10⁰³ | 1.30 × 10⁻⁰² | 4.37 × 10⁻⁰⁶ |
| RBD | B-YL | 8.92 × 10⁰² | 5.08 × 10⁻⁰² | 5.70 × 10⁻⁰⁵ |

In Table 3, Association and dissociation kinetic rate constants (kon, koff) and equilibrium dissociation constants (KD) were calculated from surface plasmon resonance (SPR) kinetic measurements for aqueous RBD, and ACE2 flowing past BYL attached to a Biacore chip. Proteins were dissolved in running buffer (10 mM HEPES, 150 mM NaCl, 3 mM EDTA, 0.005% Surfactant P20, pH 7.4) and were flowed past biotin linked B-YL peptide construct on a CSM sensor chip with a Biacore system (Methodology). Kinetic rate constants and equilibrium dissociation constants were determined from curve fitting analysis of SPR traces.

### Example 3. Surface Plasmon Resonance (SPR) Measurements of SMB and B-YL Peptide Binding to ACE-2 Domain

To better characterize the potential degree and type of interaction of the lung ACE2 receptor protein and lung surfactant peptides SMB (SEQ ID NO:7) and B-YL (SEQ ID NO:27), we used SPR to test the binding of these peptides to the recombinant human ACE2 receptor construct. The SPR binding study was accomplished by immobilizing the ligand (ACE2) on the SPR sensor surface and flowing the solute containing the SMB or B-YL peptide past the sensor-linked molecule. The binding of the solute to the sensor surface ligand results in an evanescent sensor response and is measured in response units (RU) that are proportional to the bound mass.

Representative sensor grams for the interaction of the lung surfactant SMB and B-YL peptides tested in the present study with the ACE-2 receptor protein are shown in FIG. 4. The kinetic analysis of these traces to determine relative binding (affinity) of the peptides to the expressed protein construct is summarized in Table 4. The ACE2 construct (FIG. 4) had the highest binding affinity for the SMB peptide and lowest dissociation constant (K_{D}). B-YL peptide had a lower affinity with corresponding higher K_{D} values (Table 4).

TABLE 4
Binding data of SMB and B-YL surfactant peptides with rhACE2 receptor protein derived
from experimentally determined peptide-protein interaction data using SPR.

| | ka | kd | Rmax | RI | Conc of | KA | KD | Req | Kobs |
|---|---|---|---|---|---|---|---|---|---|
| | (1/Ms) | (1/s) | (RU) | (RU) | Analyte | (1/M) | (M) | (RU) | (1/s) |
| 0.5 μg/mL SMB | 6.2 × | 6.12 × | 6.48 × | 5.89 | 1.05 × | 1.01 × | 9.87 × | 5.93 × | 7.12 × |
| to hACE2 | 10³ | 10⁻⁵ | 10³ | | 10⁻⁷ | 10⁸ | 10⁻⁹ | 10³ | 10⁻⁴ |
| 0.5 μg/mL B-YL | 2.19 × | 2.79 × | 258 | 2.12 | 1.05 × | 7.85 × | 1.27 × | 255 | 2.33 × |
| to hACE2 | 10⁴ | 10⁻⁵ | | | 10⁻⁷ | 10⁸ | 10⁻⁹ | | 10⁻³ |

In the present disclosure we examined the potential interaction of two hydrophobic lung surfactant peptide mimics of surfactant protein B, named SMB and B-YL, with the ACE-2-SARS-CoV-2 Receptor-Binding Domain. Experimental in vitro SPR measurements of the binding of the SMB and B-YL peptides to the recombinant human ACE-2 receptor protein confirm the binding of the peptides to this target domain. These results suggest that lung surfactant protein B peptide mimics can be used in the therapeutic intervention of COVID-19 infection in the lung.

It is contemplated that the observed experimental difference binding affinity of the SMB peptide compared to the B-YL construct for the ACE-1 receptor may be related to the greater rigidity of the disulfide-linked structure of the helix hairpin mimic conformation.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for preventing, inhibiting or treating the infection of a virus in a subject in need thereof, or treating the infection or an associated symptom in the subject, comprising administering to the subject a peptide comprising:
(i) a first fragment comprising the amino acid sequence of XWLXRALIKRIQAZI (SEQ ID NO: 1) or a first amino acid sequence having at least 90% sequence identity to SEQ ID NO: 1 and
(ii) a second fragment comprising the amino acid sequence of RZLPQLVXRLVLRXS (SEQ ID NO: 2) or a second amino acid sequence having at least 90% sequence identity to SEQ ID NO: 2,
wherein X is any amino acid and Z is any amino acid.

2. The method of claim 1, wherein at least one amino acid at the X positions is not cysteine.

3. The method of claim 2, wherein each amino acid at the X positions is not cysteine.

4. The method of claim 1, wherein the amino acid at each X position is selected from the group consisting of Y, L, A, and F.

5. The method of claim 4, wherein the amino acid at each X position is selected from the group consisting of Y and F.

6. The method of claim 1, wherein at least one amino acid at the Z positions is not methionine.

7. The method of claim 6, wherein each amino acid at the Z position is not methionine.

8. The method of claim 1, further comprising (iii) a turn between the first fragment and the second fragment.

9. The method of claim 8, wherein the turn comprises PKGG (SEQ ID NO:3).

10-11. (canceled)

12. The method of claim 1, wherein the first fragment is at the N-terminal end of the second fragment.

13. The method of claim 12, further comprising an insertion sequence at the N-terminal end of the first fragment.

14. The method of claim 13, wherein the insertion sequence comprises FPIPLPY (SEQ ID NO:5).

15. The method of claim 1, wherein the peptide is 100 amino acids in length or shorter.

16. The method of claim 1, wherein the first fragment comprises any amino acid sequence of SEQ ID NO:11-18, an amino acid sequence having at least 90% sequence identity to any amino acid sequence of SEQ ID NO:11-18, or an amino acid sequence derived from any amino acid sequence of SEQ ID NO: 11-18 with one, two or three amino acid addition, deletion and/or substitution.

17. The method of claim 1, wherein the second fragment comprises any amino acid sequence of SEQ ID NO:19-26, an amino acid sequence having at least 90% sequence identity to any amino acid sequence of SEQ ID NO:19-26, or an amino acid sequence derived from any amino acid sequence of SEQ ID NO: 19-26 with one, two or three amino acid addition, deletion and/or substitution.

18. The method of claim 1, wherein the peptide comprises any amino acid sequence of SEQ ID NO: 7-10 and 27-59, an amino acid sequence having at least 90% sequence identity to any amino acid sequence of SEQ ID NO: 7-10 and 27-59, or an amino acid sequence derived from any amino acid sequence of SEQ ID NO: 7-10 and 27-59 with one, two or three amino acid addition, deletion and/or substitution.

19-20. (canceled)

21. The method of claim 1, wherein the virus is a coronavirus.

22. The method of claim 21, wherein the coronavirus is SARS-CoV-2, SARS-CoV, or Middle East respiratory syndrome-related coronavirus (MERS-CoV).

23. The method of claim 1, wherein the virus is an influenza virus.

24. The method of claim 23, wherein the influenza virus is the H5N1 influenza A virus or the H7N9 influenza A virus.
